(19) United States
(12) Patent Application Publication
(10) Pub. No.: US 2012/0009560 A1
(43) Pub. Date: Jan. 12, 2012

(54) Method For Purifying Nucleic Acids From Microorganisms Present In Liquid Samples

(76) Inventors: Stéphane Coupe, Grenoble (FR); Dorothée Jary, Sassenage (FR)

(21) Appl. No.: 13/054,294

(22) PCT Filed: Jul. 16, 2009
(86) PCT No.: PCT/FR09/00872

§ 371 Date: Apr. 4, 2011

(30) Foreign Application Priority Data
Jul. 16, 2008 (FR) 0804039

(51) Int. Cl. C12Q 1/70; C12M 1/00; C12Q 1/68

(52) U.S. Cl. 435/5; 435/6.12; 435/283.1

(57) ABSTRACT

The present invention relates to a method for treating liquid samples with a view to detecting any possible pathogenic microorganisms in a very small amount. More specifically, this method comprises a generic step of capturing and concentrating microorganisms on an ion exchange surface, followed by an in situ lysis treatment carried out on the microorganisms and capture of the nucleic acids released during the lysis. The implementation of this method enables an extremely concentrated and purified solution of nucleic acids to be obtained. This method is suitable for a continuous treatment of liquid samples. The invention also relates to a device for analysing liquid samples for biology, health or the environment, which is suitable for the implementation of the method according to the invention.

The invention relates to the field of the overall detection of pathogenic microorganisms which are possibly weakly represented in the samples to be analyzed and the effect of which on public health, whether human or animal public health, can be considerable.

The present invention relates more specifically to a method for treating liquid samples with a view to detecting any possible pathogenic microorganisms in very small amounts. More specifically, this method consists of a generic step of capturing and concentrating microorganisms, followed by an in situ lysis treatment carried out on the microorganisms and capture of the nucleic acids released during the lysis. The implementation of this method makes it possible to obtain an extremely concentrated and purified solution of nucleic acids. This method is, in addition, suitable for continuous treatment of liquid samples.

It also relates to devices for analyzing liquid samples for biology, health or the environment; in particular, cellular and molecular collector-concentrator devices, and more generally integrated devices for treating samples.

### Sample Quality Problem

The samples to be treated may be of complex biological and physicochemical composition. The characteristics which make the analysis of the sample difficult lie essentially in the variability of the total biomass, in the ionic strength, in the pH, in the presence of colloids, small molecules from decomposition or of organic materials, or else artificial chemical substances in said sample.

Thus, it is common for the samples to be analyzed to contain a more or less concentrated ubiquitous microbiological flora which has no real effect on public health or on the scientific studies carried out. This is, for example, the case with samples of water (industrial, environmental) and the coliforms that they may contain, these bacteria being pathogenic only at high concentrations; this is also valid for samples obtained from air or from sludge or else fecal samples.

In many cases, it may prove necessary to search for microorganisms which are not very concentrated or are weakly represented in a sample, for example in the case of the detection of pathogens capable of colonizing beverage water circuits, industrial or environmental water, or air. These pathogens are most commonly viruses, bacteria, protozoa, amebae, fungi, yeast, algae, worms, etc. This detection is essential since these pathogens can then be responsible for serious diseases, among which are cholera, legionellosis and typhoid, in which the following symptoms may be observed: diarrhea, dysentery, gastroenteritis.

In the case of a sample which is complex owing, for example, to a very high total microbial load, or else of a sample rich in colloids, etc., the detection of a very dilute microorganism is made difficult.

### Need for Microbiological Monitoring

Given the possible contaminations of water by infectious agents, it is necessary to be able to monitor the pathogenic microorganism load with sufficient frequency.

In the case of environmental water, the controls make it possible, if necessary, to implement preventive measures in terms of human or animal frequentation, by limiting access to the sites for example, and in terms of sampling for the purposes of treatment for drinking water networks, inter alia.

In the case of industrial water networks, microbiological monitoring makes it possible to take steps to disinfect or treat the water or the industrial equipment before discharge into the environment, and thus to adhere to regulatory microbiological standards.

Monitoring of the microbiological quality of the air is subjected to the same problems with the same health consequences as those defined for water.

### Optimization of the Sample Treatment Methods

Most of the conventional techniques used for treating liquid samples of large volumes have several drawbacks. They treat isolated samples, and are not very sensitive owing to the poor purification yields and the large dilutions necessary before the biological analyses. Finally, they are lengthy, require heavy equipment and are expensive in terms of personnel and materials.

The reference technique is based on identification and counting of the microorganisms by culturing on various selective media. This is a technique which is lengthy to implement, often taking more than 24 hours, and which can be biased both by the commensal flora and also by the presence of microorganisms in growth lag, or even microorganisms which are viable but cannot be cultured but are nevertheless pathogenic. Furthermore, this method cannot take into account slow-growing microorganisms.

Another technique allows rapid quantification of the total microbial population by measuring enzyme activity. This technique has the advantage of taking into account bacteria which are viable but cannot be cultured. On the other hand, it does not allow specific identification of the microorganisms.

Finally, other techniques allow more specific answers to be obtained. This is the case of microbial capture using specific antibodies. However, the time to obtain an answer is still often long and the technique can exhibit sensitivity and specificity faults owing to cross reactions. Likewise, the use of labeled nucleic probes allows very specific detection after hybridization with their targets. The drawback here lies in the small number of targets per cell, which makes this technique relatively insensitive and unsuitable for the detection of microorganisms which are not very concentrated. Over the past few years, this drawback has been overcome by using real-time gene amplification techniques, for example by PCR or NASBA.

However, most techniques first require a laborious treatment by filtration, by centrifugation or by precipitation. However, the filters or pellets generated are, respectively, often saturated or compact, and contain many molecules and particles that are difficult to characterize, and which are known to strongly interfere with the analytical biological processes, such as antigen-antibody interactions or gene amplifications. It is therefore often necessary to very greatly dilute the samples in order to avoid inhibition of detection reactions and to avoid false-negative results.

Finally, the samples are taken from time to time, over time, with frequencies that may be variable. The results obtained therefore correspond to instantaneous images of a state of contamination of a particular environment. They cannot take into account the variabilities in contamination related to the environment.

The analysis of the studies for developing integrated systems miniaturized to the micrometric scale shows that the step of preparing the samples is crucial and very difficult to integrate, all the more so when all the preparative steps are carried out by the same microsystem, from the capture of the elements to be investigated, to the obtaining of the pure and ultraconcentrated molecules.

It therefore appears that a system capable of capturing and concentrating the microorganisms continuously over a relatively long period of time would be more informative of the state of contamination of an environment than the taking of an isolated sample, and would allow both a better concentration and a better purification. Overall, this would make it possible to be able to work with standard laboratory-on-chip systems and to avoid, for example, the dilutions necessary before the gene amplifications as carried out today.

Consequently, it is of use to develop an integrated system which meets the following criteria:

1. A broad range of use allowing simple and generic sample treatment strategies,
2. A considerable change in scale of the sample to be treated and which can be integrated into microsystems,
3. A large purification and concentration capacity so as to allow the analysis of any sample and to allow the detection of trace microorganisms (the number of which may be less than 10 in 100 ml),
4. A rapid preparation time, less than 20 minutes.

The method of the invention satisfies these various requirements.

Miniaturized devices (biochips) for detecting microorganisms have been developed. Liu et al. describe a biochip for the detection of bacteria, in which the following steps are carried out: immunomagnetic capture of the target bacteria; preconcentration of the bacteria and purification thereof; lysis of the bacteria; PCR amplification of the nucleic acids obtained, and detection based on electrochemical DNA chips (Liu et al. Anal. Chem. (2004) 76, 1824-1831). By virtue of the immunomagnetic capture of the target bacteria, this device enables only one specific detection, targeting a specific bacterium.

Cheng et al. describe a biochip for detection of bacteria in blood samples, comprising a fluidic chamber in which the following are carried out. Dielectrophoretic separation of the bacterial cells and of the blood cells; electronic lysis (by application of a series of electrical pulses) of the captured bacteria; digestion of the proteins with proteinase K; and analysis of the RNA and DNA released with DNA chips. The dielectrophoretic separation step allows the capture of only a small fraction of the bacteria, thus limiting the applications of this device; it is in particular not suitable for the treatment of samples that may contain small amounts of microorganisms (Cheng et al. Nature Biotechnology (1998) 16, 541-546).

It has been shown that certain ion exchange polymers, such as polyethyleneimine (PEI), can adsorb bacteria or viruses (Deponte et al. Anal Bioanal Chem. 2004; 379(3):419-26; Yamaguchi et al. J Virol Methods. 2003 December; 114(1):11-9); cellulose matrices bearing sulfate ester functions have also been described for the capture of viruses or viral particles (EP 1 808 607). Such ion exchange polymers also allow the capture of chemical molecules (Boom et al. J Clin Microbiol. 1990 March; 28(3):495-503; U.S. Pat. No. 5,342,931 “Process for purifying DNA on hydrated silica”; U.S. Pat. No. 5,503,816 “Silicate compounds for DNA purification”) with good effectiveness in terms of kinetics, purification and saturation.

Some systems have been developed for extracting DNA from blood samples having a volume of a few milliliters. The extracted DNA is purified and then eluted in at least 100 μl (King Fisher, Easy Mag).

However, none of these devices or methods allows at the same time a microorganism capture which is generic in nature, the possibility of lysing the microorganisms in situ and also capture of the nucleic acids released during said lysis.

The applicant has now demonstrated the possibility of applying a pretreatment to liquid samples that may contain microorganisms with a view to a subsequent analysis of the nucleic acids of said liquid samples.

The method implemented in this invention is based on the use of ion exchange surfaces for carrying out all the nucleic acid preparation steps, from the capture of the microorganisms to the purification and concentration of the nucleic acids (DNA and RNA).

To summarize, the advantages of the method and of the device described are:
a generic capture;
a high degree of concentration of the microorganisms and, optionally, of the nucleic acids;
a high degree of purification of the microorganisms and, optionally, of the nucleic acids;
simple integration in automated systems.

More specifically, the present invention relates to a method for treating a liquid sample with a view to analyzing the nucleic acids of the microorganisms that may be contained in said sample, consisting in:

(A) capturing the microorganisms contained in said liquid sample by adsorption of said microorganisms onto a first anion and/or cation ion exchange active surface;
(B) lysing said microorganisms in the presence of said first active surface onto which said microorganisms are possibly adsorbed;
(C) adsorbing the nucleic acids released during step (B) onto a second anion exchange active surface.

Preferably, the lysis step (B) is carried out in situ while said microorganisms are adsorbed onto said first active surface and/or said first active surface is an anion exchange surface and said second active surface is the same as said first active surface.

The generic nature of the method comes from its ability to be able to be implemented for all of the microorganisms defined below and the nucleic acids without distinction of particular specificity that may, a priori, differentiate them from one another.

The term “liquid sample” is intended to mean a sample taken from industrial water (for example originating from a cooling circuit), from environmental water, or else from drinking water intended for human or animal consumption and, by extension, any sample in which the element(s) to be detected is (are) in solution or in suspension. This sample may itself have been obtained from a sample taken or another sample containing the elements of interest, for example a body fluid, a sample taken from air, obtained from physical and/or chemical and/or biological treatment according to any method that can be adapted by those skilled in the art.

Preferably, the samples are aqueous samples or samples with a high aqueous component.

In order to avoid any exogenous contamination, the samples are preferably taken under extremely clean conditions with sterile material.

In the case of analysis of the microbiological quality of the air, the liquid samples are prepared according to techniques known to those skilled in the art (see, in particular, the publication by Stachowiak J C, Shugard E E, Mosier B P, Renzi R F, Caton P F, Ferko S M, Van de Vreugde J L, Yee D D, Haroldsen B L, VanderBoot V A. Autonomous microfluidic sample preparation system for protein profile-based detection of aerosolized bacterial cells and spores. Anal. Chem. 2007 Aug. 1; 79(15):5763-70).

The volume of the liquid samples is between 1 and 100 ml, it is preferably 10 ml.

Preferably, the liquid sample is such that:
it contains no ionic detergents, or a negligible amount, i.e. less than 5% by weight relative to the total weight of the sample, of ionic detergents;
its saline concentration does not exceed 2 M and is preferably less than 200 mM;
its pH does not exceed 10, its pH is preferably close to neutrality.

The term “microorganisms” is intended to mean enveloped or nonenveloped viruses, Gram-positive and Gram-negative vegetative bacteria, bacteria in sporulated form, protozoa, microscopic fungi and yeasts, microplancton, pollens, animal cells and plant cells which it is desired to capture and/or concentrate and/or purify and/or detect.

The term “ion exchange active surface” is intended to mean any more or less strong ion (anion or cation) exchange surfaces which allow the adsorption of microorganisms, or of constituents thereof down to the molecular level. Preferably, the active surface is chosen from strong ion exchange resins. Preferably, the active surface will be an anion resin or anion exchange surface.

The expression “anion resin or anion exchange surface” is intended to mean a surface bearing chemical functions which are charged in respect of the pH conditions. This is, for example, the case of quaternary amines, all the bonds of which are involved with radicals other than protons. Thus, for example, polyethyleneimine (PEI) is a strong anion exchanger since a fraction of the amines are quaternary amines, whereas diethylaminoethyl (DEAE) is a weak exchanger since no amine is quaternary. Thus, the higher the proportion of quaternary amine, the stronger the strong anion exchange surface.

Anionic resins (anion exchange) can be classified according to Table I below:

TABLE I

| | |
|---|---|
| strong | quaternary amine groups |
| | tertiary amine groups |
| weak | secondary and primary amine |
| | groups |

Cationic resins (cation exchange) can be classified according to Table II below:

TABLE II

| | |
|---|---|
| strong | sulfonic groups (SO₃⁻ |
| | group) |
| intermediate | phosphorus groups |
| weak | carboxymethyl or carboxylic |
| | groups |

The anion or cation exchange surfaces may be fixed or mobile, arranged within a device for extracting and purifying nucleic acids from microorganisms; they are chosen from charged polymers of which the branching with a carbon chain makes it possible to reinforce the charge; in particular, they are selected from the group consisting of resins having groups chosen from quaternary amine groups, tertiary amine groups, secondary amine groups, primary amine groups, sulfonic groups, phosphorus groups, carboxymethyl or carboxylic groups; or else hydroxyapatite, diethylaminoethyl (DEAE), polylysine and polyethyleneimine (PEI) resins.

Said ion, in particular anion, exchange surfaces described above allow the generic adsorption of the microorganisms, and can be adapted for a very broad spectrum of applications. In addition, the elimination of the liquid medium initially containing the microorganisms to be sought can be easily carried out without however risking the detachment of the microorganisms from the surface. The elimination of the liquid medium allows the captured microorganisms to be concentrated before lysis thereof. In particular, the volume of the sample can be reduced to a volume of between 1 and 10 μl.

In addition to allowing the capture of the microorganisms, the ion exchange surfaces that are of use for implementing the method of the invention withstand the physical, chemical or enzymatic methods for opening up the microorganisms in order to release their nucleic acids. Preferably, these surfaces also allow the purification and concentration of the nucleic acids in a solvent compatible with biological reactions, in very small volumes, from 1 to 10 μl.

The anion or cation exchange surfaces advantageously lie on a support material which allows the reception of the active groups with respect to the method described, and the integrity of which is not impaired, or impaired very little, by the treatments for opening up the microorganisms. By way of example, and in a nonlimiting manner, mention may be made of silica and polycarbonate.

The surfaces and their support may be either mobile, as in the case of magnetic beads for example, or fixed, as in the case of a laboratory-on-chip.

The term “laboratory-on-chip” is intended to mean any fluidic and/or microfluidic devices comprising adequately proportioned, structured and functionalized (by surface modification or filling with functional reactants) zones of passage of the sample in a completely or partially automated or nonautomated manner.

When the active surface and its support are mobile in the form of beads, the latter are added to the entire liquid sample and become distributed such that they behave like a net, with the mesh size being the smallest distance between the beads. The device should then be capable of collecting the beads in order to retain the captured elements (microorganisms, nucleic acids) and to concentrate the sample.

When the support is fixed, there is no risk of aggregation. The device should, however, be proportioned and designed so as to allow effective capture of the microorganisms, for example by promoting the probabilities of active surface-sample encounter.

Finally, the sample is brought into contact with the active surfaces in fractions, at speeds which depend on the flow rate applied to the device.

According to one particular embodiment of the method according to the invention, step (A) of capturing the microorganisms contained in a liquid sample is carried out on an active surface which is either an anion exchange active surface when it is a question of capturing microorganisms of which the net surface charge is negative, or a cation exchange active surface when it is a question of capturing microorganisms with a net positive surface charge. Preferably, the active surface is an anion exchange surface chosen from quaternary amine groups, tertiary amine groups, secondary amine groups, primary amine groups, or else hydroxyapatite, diethylaminoethyl (DEAE), polylysine and polyethyleneimine (PEI) resins.

Step (A) can be carried out with an ion exchange surface such that:
the active surface per unit of volume of liquid sample that can be contained in the chamber in which the microorganism capture is carried out is between 1 and 200 m²/l of sample, preferably between 9 and 100 m²/l;
the distance between the active surface and the elements to be captured is about 10 to 100 micrometers, preferably 10 micrometers;
the time for which the liquid sample is brought into contact with the active surface is about 30 seconds to 10 minutes, preferably 10 minutes;
the bringing-into-contact temperature is between 4° C. and 40° C., preferably 25° C.±5° C.

Regular stirring of the sample, for example using a vortex, can be carried out. This stirring proves to be advantageous when beads are used as active-surface support.

Following the capture, an optional concentration step (A′) is advantageously carried out by physical separation of the capture surface (fixed support or mobile support) and of the liquid solvent. In the case where the active surface is mobile and carried by magnetic beads, magnetic attraction of the beads for 30 seconds to 2 minutes, or until clarification of the sample, is recommended.

Step (B) of opening up (lysing) the microorganisms, allowing the release of the nucleic acids, is carried out directly on the microorganisms retained on the active surface; it can be carried out by any method known to those skilled in the art, in particular by sonication, abrasion using glass beads, enzyme digestion, heat shock, osmotic shock, light irradiation, electroporation, the action of microwaves, etc., according to the sample under consideration and the application envisioned following this step.

One advantage of this method is that of carrying out the lysis in situ, i.e. while the microorganisms are adsorbed on the active surface.

Making a saving in terms of a microorganism elution step is advantageous; this is because the fewer steps the method comprises, the more the transfers of liquids, which are potentially factors of contamination and of loss of material, are avoided; in addition, this makes the method easier to automate.

Concretely, step (B) of lysing the microorganisms directly in the presence of the active surface can be carried out by various chemical and/or physical and/or biological methods which do not impair, or impair very little, the structural and functional properties of the active surfaces and/or of their supports.

By way of example, the lysis can be carried out by enzymatic digestion with lysozyme alone or with lysozyme then proteinase K. Successive digestion with lysozyme and then with proteinase K will preferably be carried out under the respective lysis conditions in a medium having the composition: 50 mM Tris-HCl, 50 mM NaCl, 5 mM EDTA, pH 7.5, for 10 minutes at ambient temperature, and then in a medium having the composition: 10 mM Tris-HCl, 2 mM EDTA, pH 8.0, or 4 M NaCl, 10 mM Tris-HCl, 2 mM EDTA, 0.1% sarcosyl, pH 9.0, for 15 minutes at 70° C.

According to one variant, step (B) is carried out by ultrasonication of the sample under optimum conditions according to the geometry of the chamber containing the sample and according to whether the support of the active surface is fixed or else mobile.

According to another variant of the invention, the present method can comprise an additional step (A1), which is inserted between step (A) and step (B), of purifying the microorganisms. This purification step (A1) corresponds to a washing of the surface to which the microorganisms are attached using a solution chosen so as not to disrupt the attachment of the microorganisms to the exchange surface. By way of example, a salt-based solution can be used, the characteristics of which are dependent on the active surface used.

The term “purification” is generally intended to mean the elimination of the useless or impairing compounds which have attached to the active surfaces under the same conditions as the microorganisms or the nucleic acids of interest released during step (B), but the elution of which can be carried out with suitable solutions, without any risk of eluting, or at least very partially, said microorganisms and nucleic acids which remain adsorbed at the active surface.

The optional purification step (A1) can be carried out by incubating the active surface with a salt-based washing solution, preferably having the following composition: 0.8 M NaCl, 10 mM Tris-HCl, 15% (v/v) ethanol, pH 8.

The method according to the invention also comprises a step (C), following step (B), of adsorbing the nucleic acids released by the lysis of said microorganisms.

Thus, after the opening up or lysis of the microorganisms, the released nucleic acids are, during step (C), either immediately adsorbed onto the first active surface which allowed the capture of the microorganisms, or adsorbed onto a second anion exchange surface of electrostatic force similar to that of step (A). This makes it possible to segregate and rapidly concentrate the material of interest, in this case the nucleic acid. Preferably, the same anion exchange surface is used as first and second active surface.

An optional step (A2) of eluting the microorganisms from the first active surface can be carried out, following step (A) or step (A1) and preceding step (B), with a solution which promotes the separation of the microorganisms from the first active surface by competition with an ion of the same charge, or by chemical modification of the surface charge density of the beads and/or of the microorganisms.

Step (A2) can be carried out by incubation of the active surface with a 100 mM sodium hydroxide solution, optionally containing detergents, optionally supplemented with physical methods such as heating, stirring with a vortex or ultrasound. In this case, the microorganisms contained in the eluate may be isolated from the first active surface in order to undergo the lysis intended in step (B) or else to undergo lysis in the presence of the first active surface but without being adsorbed.

An optional step (A3), following step (A2), of regenerating the first active surface can also be carried out with a sodium hydroxide (NaOH) solution so as to continue with another cycle of capture in accordance with step (A) for a predetermined number of cycles. The regeneration of the active surface for microorganism capture can be carried out under the following conditions: incubation of the active surface with a sodium chloride solution, preferably of 100 mM, then elimination thereof or else incubation of the active surface with deionized water, and then elimination thereof.

According to one variant of step (C) of the method, the adsorption of the nucleic acids is carried out on the same ion exchange surface as that used in step (A) (first active surface) which is an anion exchange surface.

According to another variant of step (C) of the method, the adsorption is carried out on a second active surface, corresponding to an anion exchange surface of use for this step (C) of adsorbing the nucleic acids, which is a prelude to the nucleic acid purification and concentration. This anionic second active surface can contain groups bearing carbon chains of various lengths, for example from C₁ to C₁₈, but also silica, DNA, RNA and synthetic nucleic acid analogs such as PNA and LNA.

Preferably, the second active surface is the same anion exchange surface as that used in step (A).

Here again, the second surface used can lie on a support and said second active surface and its support can be fixed or mobile.

The method may comprise an optional step (C1), following step (C), of purifying the adsorbed nucleic acids by washing.

This step (C1) is carried out by adding a solution based on salts with, preferably, but not necessarily, alcohol in well-defined concentrations, depending on the active surface; it allows the elimination of a large number of chemical and biological contaminants, such as sugars, proteins, lipids or small RNAs, such that there remains only the DNAs and the majority of the RNAs of the sample.

Finally, the method according to the invention may also comprise a step (C2) of eluting the nucleic acids, which follows step (C) or step (C1).

According to the experimental conditions, this step (C2) can allow the separation of the DNAs and of the RNAs. The RNA retained can be selectively eluted by means of a saline solution having a lower concentration than that required for the DNA.

After a series of washes based on salts and, optionally, alcohol, the elution is carried out by modification of the surface charge of the active surfaces, either by adjusting the pH and/or by adjusting the ion concentration in concentration ranges compatible with biochemical and biological reactions, according to methods known to those skilled in the art.

The overall method for preparing nucleic acids on anion exchange active surfaces is particularly suitable for devices of the laboratory-on-chip type. Specifically, the surface capture makes it possible to envision a very large reduction in scale from the first steps of treatment of the sample. According to whether the active surface is borne by beads or whether it coats a fixed support, the sample of interest will in the end be confined to the capture surface, overall the microorganisms and/or DNA and/or RNA will in the end be stored in a two-dimensional, i.e. surface, structure instead of being stored in solution in a three-dimensional reservoir.

The invention may be available according to several variants; in particular, subsequent sample treatment steps can be added. Once the nucleic acids have been collected, either on an active surface or in solution, it is possible to purify them and to collect them according to a similar method which consists in adsorbing the nucleic acids onto an active surface of PEI or silanol type, according to step (D).

Step (D) of capturing the nucleic acids on an active surface, similar to or different than the first active surface for capturing the microorganisms, can be carried out under physicochemical conditions suitable for the type of active surfaces. To do this, the active surface is incubated with a solution which has a saline concentration of less than 0.8 M of NaCl, preferably less than 200 mM, which is free of detergent, and which has a pH below the pKa of the group involved in the adsorption, preferably around neutrality, in the case of an active surface similar to PEI.

Incubation of the active surface with a solution of guanidine HCl having a concentration of greater than 3 M, 10 mM Tris-HCl and 80% (v/v) ethanol, pH 4.5, in the case of a silanol active surface, is preferred.

The method according to the invention may also be supplemented with a step (E) of purifying said nucleic acids.

This nucleic acid purification step (E) is carried out by passing over a washing solution via the incubation of the active surface with a salt-based solution, preferably having the composition: 0.5 M NaCl, 10 mM Tris-HCl and 15% (v/v) ethanol, pH 8, for a surface of PEI type, or via the incubation of the active surface with a salt-based solution, preferably having the composition: 2 mM NaCl, 5 mM EDTA and 80% (v/v) ethanol, pH 7.0.

Finally, the method may also comprise a step (F) of final concentration via a drying step consisting in eliminating the residual liquids, followed by an elution in a small volume of liquid.

In step (F), the elution of the nucleic acids in a small volume is carried out using a solution which promotes the separation of the nucleic acids from the active surface, according to the same principles as those described in (A2); with, preferably, incubation of the active surface with a solution of NaOH which does not exceed 100 mM, preferably 50 mM, for 20 seconds at ambient temperature, for an active surface of PEI type, or incubation of the active surface with a solution containing 10 mM Tris-HCl, at 60° C. for 2 minutes, for a silanol-type surface.

According to another of its aspects, the invention relates to a device for treating a liquid sample that may contain microorganisms, characterized in that it contains at least one ion, preferably anion, exchange surface, as defined above, placed in a chamber such that the ratio of said active surface per unit volume of sample which can be contained in said chamber is between 1 and 200 m²/l, preferably between 9 and 100 m²/l.

According to one particular embodiment of said device, it comprises a laboratory-on-chip consisting of mobile beads supporting the active surfaces. In this scenario, the optimum inter-bead distance for the microorganism capture step is approximately 10 μm. This distance is also optimal for the nucleic acid capture. In addition, the surfaces produced which are given per unit volume on the basis of kinetic studies of microorganism capture (see the theoretical model of Deponte et al.) correspond well to the experimental data obtained.

Those skilled in the art can then proportion a laboratory-on-chip on the basis of the prior art data (for example, Microfabricated reaction and separation systems, Madhavi Krishnan et al. in “Analytical Biotechnology”; published by Elsevier Science).

Firstly, these three data elements should be defined:
The surface per unit volume (S/V) ratio of the chamber through which the sample will pass is calculated with the value of the ion exchange active surface present in the chamber and the value of the volume of sample which can be contained in the chamber;
The volume of sample that can be contained in the capture chamber; and
The flow rate of the sample.

Each input data element influences the other two.

By way of example, when a chamber is used which has dimensions of 20 mm×5 mm×0.01 mm; the volume of said chamber is 1 μl and the surface of the two rectangles (20 mm×5 mm)=200×10⁻⁶ m², then the S/V ratio is 200 m²/l, which, as things stand, is much greater than the active surface developed by the 1 μm beads used in the description of the method which follows, and can therefore bring even more effectiveness to the method.

The active surface may also be increased by adding structuring of the chamber, for example by means of pillars, or else fluidic circuit parallelization.

According to the kinetics model of Deponte et al. (see above), the adsorption speeds should be very rapid and therefore allow high flow rates. Suitable proportioning and structuring of the chambers containing the active surface in contact with the liquid sample should therefore make it possible to treat volumes of samples with, at least, the same performance levels as those described in the examples presented hereinafter.

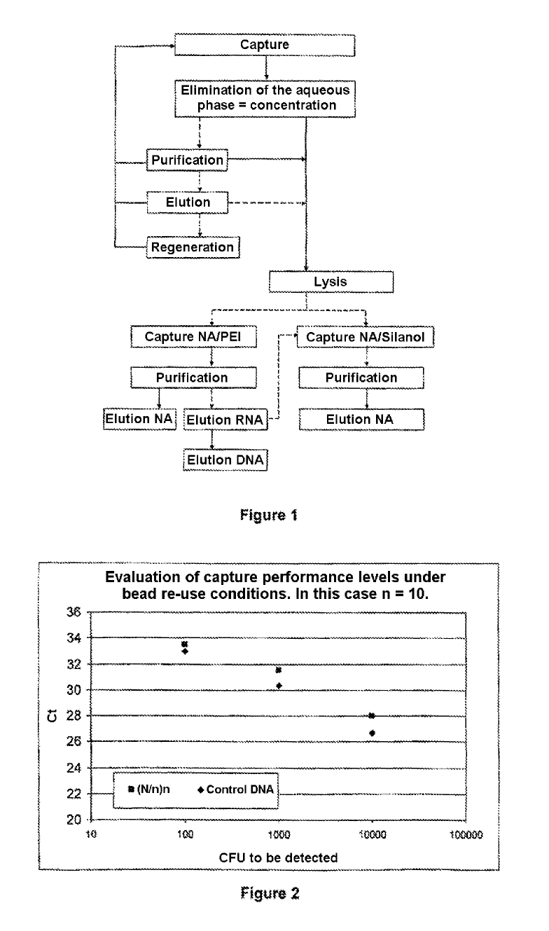

In accordance with FIG. 1, a method for preparing nucleic acids according to the invention is described for the microorganisms present in liquid samples. In this FIG. 1, the dashed arrows represent optional steps, or else alternative possibilities for treating the sample; the term “NA” means nucleic acids. There are as many possible sample preparation variants as there are combinations of pathways represented by the figure.

This invention finds application in very varied fields:
any biological analyses where the elements sought are very dilute and where the entire sample must be analyzed;
in fluidic microsystems (laboratory-on-chip (LOC) or total analysis microsystem (μTAS)), in which the integration of modified surfaces (fixed or mobile of magnetic bead type) is controlled, and
in all integrated systems requiring sample preparation, nucleic acid extraction-purification type.

This sample preparation method described in the present invention offers several advantages:
the simple and generic capture of microorganisms without size distinction;
the capture of microorganisms and of the constituents thereof even at very low concentrations;
the capture of microorganisms and of constituents thereof, even when very concentrated;
the capture of weakly concentrated microorganisms included in a high concentration of total biomass;
the preparation of a liquid sample with a high concentration of microorganisms, making it possible for the method to be incorporated in laboratory-on-chip devices;
the continuous capture and concentration of the liquid sample, which may be the whole sample or else a modified or unmodified fraction of the whole sample;
the possibility of purifying the captured microorganisms;
the possibility of eluting and regenerating the active surfaces, making it possible to treat large volumes of samples (by modified or unmodified fractions);
the possibility of lysing the microorganisms in the presence of the active surface;
the capture and purification of the nucleic acids;
the possibility of differential elution of the DNA and of the RNA;
the high concentration of the nucleic acids;
the implementation of the method in a short period of time, about 15 to 20 minutes.

The invention is now described in greater detail in relation to the following figures:

FIG. 1 shows a general flowchart detailing the various steps of the method according to the present invention; it is the method used in example 1.

FIG. 2 details the capture and regeneration performance levels of the active surfaces according to the invention.

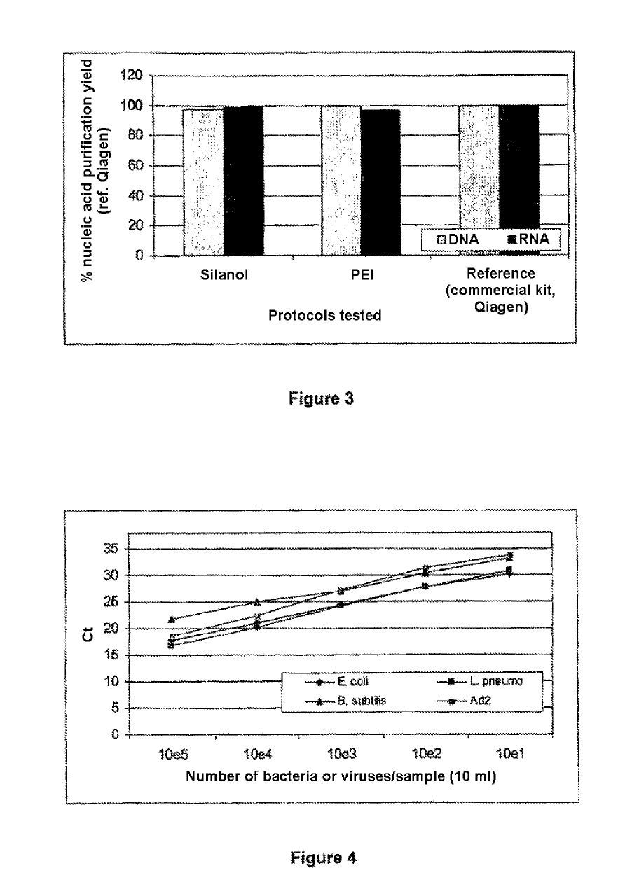

FIG. 3 shows the capacity in terms of DNA or RNA sample purification yield according to the nature of the surfaces used, reference made to a commercial method.

FIG. 4 shows the detection of the model microorganisms used after capture.

### EXAMPLE 1

### Method for Preparing Nucleic Acids

### 1. Materials and Methods

The method according to the invention was tested in the preparation of nucleic acids from model microorganisms, in this case Escherischia coli and Bacillus subtilis for the vegetative bacterial forms, Bacillus subtilis for the sporulated bacterial forms, human adenovirus type 2 (group C) for the viruses, and Cryptosporidium parvum for the protozoa, using an active surface coated with polyethyleneimine (PEI) for the capture-concentration and purification of the microorganisms and possibly of the nucleic acids, and with silanol for the nucleic acids.

The experimental protocol is described generically for all the microorganisms tested.

### 1-A. Protocol of Step (A)

A liquid sample of 10 milliliters is precollected; it may be the whole sample, optionally modified for example by means of a treatment such as an ultrafiltration, or else a fraction of the total sample, optionally modified for example by means of a treatment such as an ultrafiltration.

This sample is brought into contact with a polyethyleneimine (PEI) active surface, in this case 9.2 m²/liter of sample, supported by superparamagnetic beads one micrometer in diameter (Chemicell). The bringing into contact is carried out for 10 minutes with stirring using a vortex, in order to keep the beads well dispersed, and at ambient temperature. The beads are collected using a magnet until the sample is clarified, and then the liquid phase is eliminated.

### 1-B. Protocol of Step (B)

According to step (B), the beads, containing the microorganisms at their surface, are subjected to a lysis step in order to allow said microorganisms to be opened up and the nucleic acids to be released.

According to one variant, the microorganisms adsorbed to the beads are purified with 500 μl of a solution of 0.5 M NaCl, 10 mM Tris-HCl, 15% (v/v) ethanol, pH 8.0 before being lysed (A1).

According to one variant, the purified microorganisms are eluted with a 100 mM NaOH solution at ambient temperature for two minutes, and then separated from the active surface (in this case supported by the beads) so as to be stored (A2).

Finally, according to one variant, after elution of the microorganisms, the active surface can be regenerated by adding 100 mM NaOH and then deionized water, and returns to the initial capture step (A3).

### 1-B.1. Lysis Under “Low Salt” Conditions

The microorganisms are lysed in the presence of the active surface supported by the beads, containing 200 μl of a solution of 50 mM Tris-HCl, 50 mM NaCl, 5 mM EDTA, pH 7.5, termed “low salt”.

### 1-B.2. Lysis Under “High Salt” Conditions

According to one variant, the beads can be contained in 200 μl of a solution of 4 M NaCl, 50 mM Tris-HCl, 5 mM EDTA, 0.1% sarcosyl, pH 8.5, termed “high salt”. The lysis is carried out by ultrasonication.

According to another variant, the lysis is carried out by means of two successive enzymatic digestions (with lysozyme and proteinase K) as described above.

### 1-C. Protocol of Step (C)

### 1-C.1. “Low Salt” Conditions

In the case of a lysis carried out under “low salt” conditions, the beads which have adsorbed the nucleic acids are collected and the aqueous phase is eliminated. The nucleic acids adsorbed to the PEI are purified with a solution of 0.5 M NaCl, 10 mM Tris-HCl, 15% (v/v) ethanol, pH 8.0, according to step (C1). The beads are collected and the purification solution is expelled. The nucleic acids are then eluted by incubating the PEI beads in 100 μl of a 100 mM NaOH solution at ambient temperature for 10 seconds. The beads are collected, the nucleic acids are recovered, and the 100 mM NaOH solution is neutralized by adding 100 μl of a 100 mM HCl solution according to step (C2). The nucleic acids in solution are then brought into contact with the active surface, in this case 1.18 m²/l of sample, for 30 seconds with stirring at ambient temperature, according to (D). The beads are collected and the aqueous phase is eliminated. A purification step according to (E) is carried out by incubating the beads in a solution of 0.5 M NaCl, 10 mM Tris-HCl, 15% (v/v) ethanol, pH 8.0. Finally, an elution step is carried out with 2 to 10 μl of a 100 mM NaOH solution at ambient temperature for 10 seconds. The beads are collected, and the supernatant is recovered and neutralized by adding the same volume of 100 mM HCl, according to step (F).

According to one variant, the nucleic acids eluted from the PEI beads at the end of the lysis step and neutralized can be mixed with five volumes of solution of 3 M guanidine HCl, 20 mM Tris-HCl, 80% (v/v) ethanol, pH 4.5. Beads one micrometer in diameter producing a silanol active surface of 9.2 m²/l of sample are then added according to (D). The beads are collected and the aqueous phase is eliminated. The adsorbed nucleic acids are purified twice by adding 500 μl of a solution of 2 mM NaCl, 10 mM Tris-HCl, 75% (v/v) ethanol, according to (E). The beads are collected and the aqueous phase is eliminated. The nucleic acids are then eluted in 5 to 10 μl of 10 mM Tris-HCl, pH 8.0, with stirring and at 60° C., in accordance with step (F).

### 1-C.2. “High Salt” Conditions

In the case of a lysis carried out under “high salt” conditions, the experimenter collects the beads which have adsorbed the nucleic acids and recovers the aqueous phase, and mixes said aqueous phase with five volumes of solution of 3 M guanidine HCl, 20 mM Tris-HCl, 80% (v/v) ethanol, pH 4.5. Beads one micrometer in diameter producing a silanol active surface of 0.92 m²/l of sample are then added according to (D). The beads are collected and the aqueous phase is eliminated. The adsorbed nucleic acids are purified twice by adding 500 μl of a solution of 2 mM NaCl, 10 mM Tris-HCl, 75% (v/v) ethanol, according to step (E). The beads are collected and the aqueous phase is eliminated. The nucleic acids are then eluted in 5 to 10 μl of 10 mM

Tris-HCl, pH 8.0, with stirring and at 60° C., in accordance with the principle of concentration in a very small volume of step (F).

### 2. Results

The variants described above were each validated independently.

### 2-A. Capture and Elution of the Model Microorganisms

Table III collates the results of elution of the model microorganisms (B. s for Bacillus subtilis, E. c for Escherichia coli, Cp for Cryptosporidium parvum and Ad2 for human adenovirus type 2).

TABLE III

| Solutions used | Log₁₀ reduction | | | |
|---|---|---|---|---|
| (Conditions) | B.s | E.c | Cp | Ad2 |

| | | | |
|---|---|---|---|
| PBS | <0.05 | <0.05 | <0.05 |
| H₂O (di) | | | |
| PBS, 0.1% SDS | <0.05 | 0.18 | <0.05 |
| PBS, 0.5% SDS | <0.05 | 0.27 | <0.05 |
| PBS, 1% SDS | <0.05 | 0.52 | 0.21 |
| 1M NaCl, pH9 | <0.05 | 3.16 | 3 |
| 2M NaCl, pH9 | 0.31 | 0.2 | 0.15 |
| 3M NaCl, pH9 | <0.05 | 0.17 | <0.05 |
| 4M NaCl, pH9 | <0.05 | 0.22 | 0.05 |
| 50 mM NaOH | >2 | 1.04 | 0.84 |
| 100 mM NaOH | >3 | >2 | >2 |
| 200 mM NaOH | >3 | >2 | >2 |
| 100 mM NAOH, | >3 | >2 | >2 |
| 0.01% to 1% SDS | | | |

This Table III shows that the PBS and H₂O conditions do not make it possible to elute the species pre-adsorbed at the active surface.

The condition most favorable to the elution is an incubation in a 100 mM NaOH solution, carried out in this case by incubating the active surfaces for two minutes at ambient temperature (25° C.) and with stirring (650 rpm).

### 2-B. Re-Use of the Beads

The capture capacity after purification, elution of the microorganisms and regeneration of the active surface (polyethyleneimine (PEI) at 9.2 m²/liter of sample, supported by superparamagnetic beads 1 μm in diameter (Chemicell)) was evaluated after ten cycles of the method comprising the purification/elution/regeneration steps; the graph for this experiment is represented in FIG. 2.

This graph shows, on three separate samples, that the capture of N microbial elements is entirely possible by means of the capture of N/10 microbial elements repeated ten times.

This test confirms that a device suitable for implementing the method according to the invention can be re-used after regeneration of the active surface.

### 2-C. Enzymatic and Physical Lysis in the Presence of the Active Surfaces

The tests carried out also show that the lysis of the microorganisms does not affect the functionality of the active surfaces.

### 2-D. DNA and RNA Adsorption

The result obtained in the following point implicitly demonstrates the ability of the active surface to suitably capture DNA and RNA.

2-E. Purification and Elution of DNA and of RNA on an Active Surface (Silanol or PEI)

The nucleic acids (NA) were prepared according to two sample preparation variants: using PEI only, or PEI+silanol. The NA eluted at the end of each of the two variants were purified once again on a Qiagen column dedicated to the purification either of DNA or of RNA on a silica column. The amount of RNA or of DNA was measured by spectrophotometry and the yield was calculated relative to the reference.

The graph in FIG. 3 shows that the sample preparation method described here allows good nucleic acid (NA) purification, in particular using the same ion exchange surface for capturing both the microorganisms and the nucleic acids.

### 2-F. Evaluation of the Complete Method

Finally, the complete method was validated under artificial and real conditions by means of the detection of the model microorganisms in various real samples.

The graph in FIG. 4 shows the detection of the nucleic acids of the model microorganisms by PCR.

At each amplification cycle, the initial copy number is doubled. The increase in the number of copies is followed in real time by measuring the specific increase in fluorescence released during the reaction. The Ct is directly proportional to the concentration of targets in the amplification reaction medium and corresponds to the first cycle of the linear phase of the amplification. It is determined automatically by the software associated with the thermocycler (Stratagene).

### EXAMPLE 2

### Device Implementing the Method Described

### Principle of the Test

The method described was optimized with magnetic beads as support of the active surfaces dedicated to the capture of microorganisms and then nucleic acids. It was validated on recognized model microorganisms: gram-negative and gram-positive bacteria, sporulated bacteria, viruses and protozoa.

It was validated on various types of liquid samples: industrial water, samples taken from the air (4 m³) containing various pollutants, such as dust or hair, and samples taken from human cervixes, artificially given a strong bacterial contamination.

### Device

The device comprises:
a peristaltic pump for moving the sample and all the liquid reagents;
a magnet, which makes it possible to collect the magnetic beads;
a heating ultrasonic device, which makes it possible to resuspend the beads collected by the magnet and to open up the microorganisms;
bead segregation treatment zones;
a computer, which makes it possible to control the steps.

### Protocol

The method was carried out in accordance with the method described in example 1 according to all the variants which are explained therein.

1. A method for treating a liquid sample with a view to analyzing the nucleic acids of the microorganisms that may be contained in said sample, characterized in that it comprises at least the following steps:
(A) capturing the microorganisms contained in said liquid sample by adsorption of said microorganisms onto a first anion exchange active surface;
(B) lysing said microorganisms in the presence of said first active surface on which said microorganisms are possibly adsorbed; and
(C) adsorbing the nucleic acids released during step (B) onto said first anion exchange active surface.

2. The method as claimed in claim 1, characterized in that lysis step (B) is carried out in situ while said microorganisms are adsorbed onto said first active surface.

3. The method as claimed in claim 1, characterized in that said first active surface is chosen from the group consisting of resins having groups chosen from quaternary amine groups, tertiary amine groups, secondary amine groups, primary amine groups; and hydroxyapatite, diethylaminoethyl (DEAE), polylysine and polyethyleneimine (PEI) resins.

4. The method as claimed in claim 1, characterized in that said first surface is on a support chosen from silica and polycarbonate.

5. The method as claimed in claim 1, characterized in that the lysis of step (B) is carried out by sonication, abrasion using glass beads, enzymatic digestion, heat shock, osmotic shock, light irradiation, electroporation and/or the action of microwaves.

6. The method as claimed in claim 1, characterized in that it also comprises a step (A1) of purifying the microorganisms by washing said first surface used previously in step (B).

7. The method as claimed in claim 1, characterized in that it comprises, prior to step (B), a step (A2) of eluting said microorganisms.

8. The method as claimed in claim 7, characterized in that it comprises, after step (A2), a step (A3) of regenerating said first active surface.

9. The method as claimed in claim 1, characterized in that it comprises, after step (C), a step (C1) of purifying the adsorbed nucleic acids by washing with a salt-based solution.

10. The method as claimed in claim 1, characterized in that it comprises, after step (C) or (C1), a step (C2) of eluting said nucleic acids by modifying the charge of said second active surface.

11. The method as claimed in claim 10, characterized in that it comprises, following step (C2), the following additional steps: step (D) of capturing the nucleic acids on a surface chosen from PEI or silanol and step (E) of purifying said nucleic acids and step (F) of concentrating said nucleic acids by elimination of the liquids and elution of said nucleic acids.

12. An integrated device for treating a liquid sample that may contain microorganisms, characterized in that it contains at least one anion exchange active surface chosen from the group consisting of resins having groups chosen from quaternary amine groups, tertiary amine groups, secondary amine groups, primary amine groups; and hydroxyapatite, diethylaminoethyl (DEAE), polylysine and polyethyleneimine (PEI) resins; said active surface being placed in a chamber such that the ratio of said active surface area per unit volume of sample which can be contained in said chamber is between 1 and 200 m²/l.
